(19) United States
(12) Patent Application Publication
(10) Pub. No.: US 2003/0130629 A1
(43) Pub. Date: Jul. 10, 2003

(54) Personal Bede-Enema

(76) Inventors: Yuriy Cherepanov, New York, NY (US); Iouri Jouk, New York, NY (US); Iouri Jouk, New York, NY (US); Yuriy Cherepahov, New York, NY (US)

Correspondence Address: Yuriy Cherepanov, 2317 First AVE # 2A, New York, NY, 10035, US

(21) Appl. No.: 09/716,097

(22) Filed: Jun. 11, 2001

Related U.S. Application Data
(60) Provisional application No. 60/167,031, filed on Nov. 23, 1999.

(51) Int. Cl. A61M 1/00

(52) U.S. Cl. 604/317

(57) ABSTRACT

Personal Bede Enema is used for washing away feces and cleaning the intestines of a body.

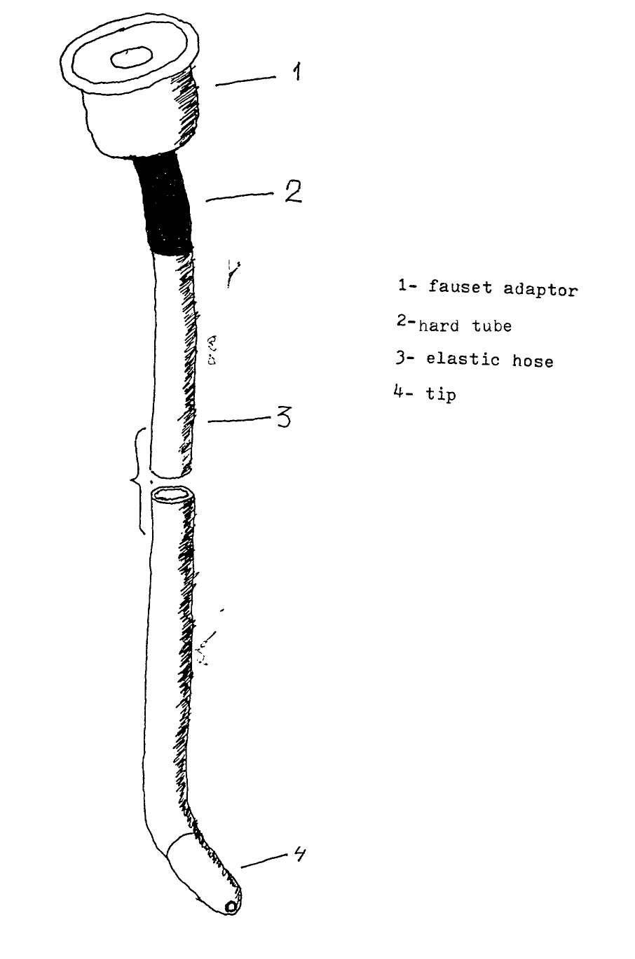

Personal Bede Enema includes a soft and flexible plastic or rubber tube of varying lengths and diameters, depending on the user.

At one end it includes a speed adapter to connect it to the tap of a local water supply system. At the other end, the tube has an oval tip with a hole for irrigation of water.

The key idea is to apply pressure from the local water supply system.

In sum, Personal Bede Enema is very simple and convenient in use for personal care.

### BRIEF SUMMARY OF THE INVENTION

The general idea of invention is:

used for washing out and washing away of bodies perineum and thick intestines of a body of the person, individually directly from a city or local water supply system pure(clean) warm water suitable for drink.

Invention is personal bede-enema.

Personal bede-enema it is a plastic or rubber soft of tube of necessary lenght and diameter.

At one end the tube has a special adapter through which it is connected to the water crane-amalgamator of a city or local water supply system. At the other end the tube has an oval tip with hole for the expiration of water.

Personal bede-enema it is used for washing genitalis of a body of the person pure, warm water under pressure it is direct from water system.

Personal bede enema, is individual means for support of hygiene.

It is soft elastic hose with fauset adaptor and hard tube one end and tip on other end

1—fauset adaptor

2—hard tube

3—elastic hose

4—tip

We claim as our invention is:

1. “PERSONAL BEDE_ENEMA”
